(19) United States
(12) Patent Application Publication
(10) Pub. No.: US 2022/0323333 A1
(43) Pub. Date: Oct. 13, 2022

(54) Deodorant Compositions

(71) Applicant: Colgate-Palmolive Company, New York, NY (US)

(72) Inventors: Shyamala Pillai, Hillsborough, NJ (US); Darrick CARLONE, New Vernon, NJ (US); Melissa MOY, Staten Island, NY (US); Stanislav JARACZ, Somerset, NJ (US)

(73) Assignee: Colgate-Palmolive Company, New York, NY (US)

(21) Appl. No.: 17/309,195

(22) PCT Filed: Nov. 8, 2018
(86) PCT No.: PCT/US2018/059798

§ 371 Date: May 5, 2021

(51) Int. Cl. A61K 8/86; A61Q 15/00; A61K 8/25; A61K 8/39

(52) U.S. Cl. CPC A61K 8/86; A61Q 15/00; A61K 8/25; A61K 8/39; A61K 2800/30; A61K 2800/34

(57) ABSTRACT

Described herein are deodorant compositions comprising an odor-reducing agent comprising a non-aluminum compound, an emollient comprising a non-volatile ether compound, calcium silicate, and an emulsifier. Methods of making and using these compositions are also described herein.

### BACKGROUND

Personal care compositions, specifically deodorant compositions, are used by rubbing an area of the body, such as the underarm, to apply a layer of the composition to the skin, and thereby reduce odor and/or perspiration. Roll-on and gel deodorants composition ideally possess the esthetic properties of smoothness, non-oiliness and non-tackiness. A roll-on antiperspirant may also be difficult to formulate and manufacture because the composition requires a sufficient viscosity to adhere to the skin, resists dripping off or running down the skin, and yet is not tacky or sticky. There is difficulty in using specific components to provide the desired esthetic and viscosity characteristics while also providing an overall composition that is stable and not at risk to phase separation between applications. For example, other compositions often require shaking prior to each use in order to re-disperse the various components throughout the composition that have phase-separated. Compositions that do not require shaking prior to each use may be too thick or have a relatively high percentage of suspending agents, thereby making such composition inappropriate for roll-on or gel deodorant applications. Therefore, there is a need for a new deodorant composition that overcomes these obstacles while still exhibit superior phase-separation stability.

### BRIEF SUMMARY

The present invention includes a deodorant composition comprising an odor-reducing agent comprising: a non-aluminum compound; an emollient comprising a non-volatile ether compound; calcium silicate; and an emulsifier.

In other embodiments, the present invention includes a deodorant composition comprising: an odor-reducing agent; an emollient that is substantially free of cyclomethicone and dimethicone; an emulsifier comprising a blend of two or more different stearyl ether; and wherein the emulsifier is present in an amount ranging from about 5.5 wt. % to about 8.0 wt. % based on the total weight of the deodorant composition.

Other embodiments of the present invention include: a method of forming a stable personal care composition, the method comprising: a) forming a composition comprising an odor-reducing agent comprising a non-aluminum compound; an emollient that is substantially free of cyclomethicone and dimethicone; and an emulsifier comprising a blend of a first stearyl ether and a second stearyl ether, wherein the first and second stearyl ethers are different and the first stearyl ether is present in an amount greater than the second stearyl ether, and the emulsifier is present in an amount ranging from about 5.5 wt. % to about 8.0 wt. % based on the total weight of the blend; wherein the composition has a viscosity ranging from about 1000 mPas to about 6000 mPas; and b) placing the formula in an analytical centrifuge and gradually increasing the separation stress from about 1000 RPM (145 G) to about 4000 RPM (2200 G) while maintaining a constant temperature of 40° C. In some embodiments, when the composition does not separate until after an equivalent of about 3500 RPM (1600 G) is applied, the formula is considered stable.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Unless otherwise specified, all percentages and amounts expressed herein and elsewhere in the specification should be understood to refer to percentages by weight. The amounts given are based on the active weight of the material. According to the present application, the term “about” means +/−5% of the reference value. According to the present application, the term “substantially free” less than about 0.1 wt. % based on the total of the referenced value.

It is contemplated that the invention described herein is not limited to the particular methodology, protocols, and reagents described as these may vary. It is also to be understood that the terminology used herein is for the purpose of describing particular embodiments only and is not intended to limit the scope of the present invention in any way.

Unless defined otherwise, all technical and scientific terms used herein have the same meanings as commonly understood by one of ordinary skill in the art to which this invention belongs. All patents, patent applications, publications, and other references cited or referred to herein are incorporated by reference for all purposes.

As used herein and in the appended claims, the singular forms “a”, “an”, and “the” include plural reference unless the context clearly dictates otherwise.

The present invention is directed to a personal care product comprising a personal care composition. In non-limiting embodiments, the personal care product may be an off-the-shelf deodorant product comprising a container that houses the personal care composition, whereby the personal care composition being a deodorant composition. As discussed in greater detail herein, the deodorant composition may be a roll-on deodorant composition. The container may comprise a housing and an applicator head. The housing may be hollow, thereby forming a reservoir for storing the deodorant composition, and the applicator head may be configured to transfer the deodorant composition contained within the reservoir to external the housing for delivery to a user's skin surface. The applicator head may be a ball rotatably coupled to the housing such that at least a portion of the applicator head is exposed to both the reservoir external the housing. The container may further comprise a cap that attaches to the housing, thereby concealing the applicator head during storage and non-use.

During use, the container may be oriented such that the deodorant composition contacts at least a portion of the applicator head, thereby coating that portion with the deodorant composition. With the cap removed, the applicator head may be contacted with the user's skin (e.g., arm pit) and the applicator head may roll relative to the housing. As the ball of the applicator head rolls relative to the housing, the previously applied deodorant composition may be exposed to external to the housing and transform the from applicator head to the user's skin.

In other embodiments, the personal care product may be an off-the-shelf deodorant product comprising a gel-deodorant. In such embodiments, the container may comprise a housing and an applicator head, whereby the housing may be hollow, thereby forming a reservoir for storing the gel deodorant composition, and the applicator head may be configured to transfer the deodorant composition contained within the reservoir to external the housing for delivery to a user's skin surface. The applicator head may be a porous face that is attached the housing such that at least one pathway fluidly couples the reservoir to external the housing. The container may further comprise a cap that attaches to the housing, thereby concealing the applicator head during storage and non-use.

During use, the container may further contain a user controlled piston at the bottom of the reservoir to drive the deodorant composition toward the applicator head, thereby pushing the deodorant composition through the pathways to deliver the deodorant composition from inside the reservoir to external the housing. With the cap removed, the applicator head may be contacted with the user's skin (e.g., arm pit) and the gel-deodorant coated applicator head may be slide against the user's skin, thereby transferring the deodorant composition from the container to the user's skin.

The deodorant composition of the present invention may be liquid or semi-liquid (i.e., gel) composition. The deodorant composition may have a viscosity at room temperature that ranges from about 1000 mPas to about 6000 mPas—including all viscosities and sub-ranges there-between. In the embodiments where the deodorant composition may be a liquid, the deodorant composition may have a viscosity at room temperature that ranges from about 1500 mPas to about 4000 mPas—including all viscosities and sub-ranges there-between. In the embodiments where the deodorant composition may be a gel, the deodorant composition may have a viscosity at room temperature that ranges from about 1500 mPas to about 7500 mPas—including all viscosities and sub-ranges there-between. The liquid deodorant composition may be suitable as a roll-on deodorant composition. The semi-liquid deodorant composition may be suitable as a gel-deodorant composition.

The deodorant composition of the present invention may comprise an emollient, an odor-reducing agent, a liquid carrier, and an emulsifier. In some embodiments, the deodorant may further comprise a preservative. In some embodiments, the deodorant may further comprise a drying agent.

The emollient of the present invention may comprise non-volatile compounds. The emollient may be an organic compound—i.e., free of compounds comprising inorganic containing groups, such as silicon oils. pounds. The emollient may be substantially free of volatile silicone containing compounds, such as volatile silicone oils that include cyclic and linear polydimethylsiloxanes, low molecular weight organo-functional silicones, and the like. In some embodiments, the emollient may be substantially free of volatile compounds.

Cyclic volatile silicones (cyclomethicones) typically contain about 3 to about 7 silicon atoms, alternating with oxygen atoms, in a cyclic ring structure. Each silicon atom is typically substituted with two alkyl groups, such as, for example, methyl groups. Volatile linear polydimethylsiloxanes (dimethicones) typically contain about 2 to about 9 silicon atoms, alternating with oxygen atoms in a linear arrangement. Each silicon atom is also substituted with two alkyl groups (the terminal silicon atoms are substituted with three alkyl groups), such as, for example, methyl groups. The linear volatile silicones typically have viscosities of less than about 5 cP at 25° C., while the cyclic volatile silicones typically have viscosities of less than about 10 cP at 25° C. “Volatile” means that the silicone has a measurable vapor pressure, or a vapor pressure of at least 2 mm of Hg at 20° C. Non-volatile silicones have a vapor pressure of less than 2 mm Hg at 20° C. Exemplary volatile cyclomethicones are D4 cyclomethicone (octamethylcyclotetrasiloxane), D5 cyclomethicone (decamethylcyclopentasiloxane), D6 cyclomethicone, and blends thereof (e.g., D4/D5 and D5/D6).

According to the present invention, emollient may be one or more compounds of an ether of stearyl alcohol, such as polypropylene glycol stearyl ether. Non-limiting examples of emollient include PPG-15 stearyl ether, PPG-5 Laureth 5, PPG-11 stearyl ether, PPG-3 myreth-3, PPG-3 myristyl ether or other propoxylated/ethoxylated fatty alcohol ethers. In a preferred embodiment, the polypropylene glycol stearyl ether is PPG-15 stearyl ether.

The emollient may be present in an amount ranging from about 3% to about 10% based on the total weight of the deodorant composition—including all amounts and sub-ranges there-between. In a preferred embodiment, the emollient may be present in an amount ranging from about 4% to about 9% based on the total weight of the deodorant composition—including all amounts and sub-ranges there-between.

The odor-reducing agent of the present invention may comprise one or more non-aluminum containing compounds. The odor-reducing agent of the present invention may comprise one or more fragrances or perfumes. The odor-reducing agent may be substantially free of aluminum-containing compounds.

Non-limiting examples of such aluminum-containing compounds include aluminum chloride, aluminum chlorohydrate, aluminum dichlorohydrate, aluminum sesquichlorohydrate, aluminum chlorohydrex polyethylene glycol, aluminum chlorohydrex propylene glycol, aluminum dichlorohydrex polyethylene glycol, aluminum dichlorohydrex propylene glycol, aluminum sesquichlorohydrex polyethylene glycol, aluminum sesquichlorohydrex propylene glycol, aluminum zirconium trichlorohyrate, aluminum zirconium tetrachlorohyrate, aluminum zirconium pentachlorohyrate, aluminum zirconium octachlorohyrate, aluminum zirconium chlorohydrex glycine, aluminum zirconium trichlorohydrex glycine, aluminum zirconium tetrachlorohyrex glycine, aluminum zirconium pentachlorohyrex glycine, aluminum zirconium octachlorohyrex glycine, potassium alum, potassium aluminum polyacrylate, and mixtures thereof.

The odor-reducing agent may be present in an amount ranging from about 0.15% to about 1% based on the total weight of the deodorant composition—including all amounts and sub-ranges there-between. In a preferred embodiment, the odor-reducing agent may be present in an amount ranging from about 0.2% to about 0.75% based on the total weight of the deodorant composition—including all amounts and sub-ranges there-between.

The deodorant composition of the present invention may comprise a liquid carrier. The liquid carrier may be aqueous. In other embodiments, the liquid carrier may be anhydrous. Non-limiting examples of liquid carrier include water and/or one or more suitable solvents, such as ethanol, isopropanol, optionally in mixtures with polyols such as propylene-, butylene-, hexylene glycol, sorbitol, glycerol, polyethylene glycol. In a preferred embodiment, the liquid carrier is water.

The liquid carrier may be present in an amount ranging from about 60% to about 90% based on the total weight of the deodorant composition—including all amounts and sub-ranges there-between. In a preferred embodiment, the liquid carrier may be present in an amount ranging from about 70% to about 85% based on the total weight of the deodorant composition—including all amounts and sub-ranges there-between.

The deodorant composition of the present invention may comprise an emulsifier. The emulsifier may be selected from a water-in-oil emulsifier, an oil-in-water emulsifier, and mixtures thereof. The emulsifier may be a non-ionic emulsifier or mixture of such emulsifiers forming an emulsifier system.

The emulsifier may comprise a hydrophilic moiety provided by a polyalkylene oxide (polyglycol), and a hydrophobic moiety provided by an aliphatic hydrocarbon, preferably containing at least 10 carbons and commonly linear. The hydrophobic and hydrophilic moieties can be linked via an ester or ether linkage, possibly via an intermediate polyol such as glycerol.

The hydrophobic aliphatic substituent may contain at least 12 carbons, and is derivable from lauryl, palmityl, cetyl, stearyl, olearyl and behenyl alcohol, and especially cetyl, stearyl or a mixture of cetyl and stearyl alcohols or from the corresponding carboxylic acids. In a preferred embodiment, the emulsifier may comprise a polyalkylene oxide ether. The polyalkylene oxide may be selected from polyethylene oxide and polypropylene oxide or a copolymer of ethylene oxide and comprises a polyethylene oxide. The number of alkylene oxide and especially of ethoxylate units within suitable emulsifiers is often selected within the range of from 2 to 100—including all ethoxylate units and sub-ranges there-between—.e.g., steareth-2, steareth-21, steareth-50, etc.

In some embodiments, the emulsifier system may comprise a first emulsifier and a second emulsifier, whereby the first and second emulsifiers are different. The first and second emulsifier may be both based on the same hydrophobic aliphatic substituent—for example, both based on stearyl alcohol—but have differing polyalkylene oxide chains. In a non-limiting embodiments, the first emulsifier may be formed from stearyl alcohol and polyethylene oxide and the second emulsifier may be formed from stearyl alcohol and a copolymer of polyethylene oxide and polypropylene oxide. In another embodiments, the first and second emulsifiers may both be formed from stearyl alcohol and polyethylene oxide, whereby the first emulsifier has a relatively shorter polyethylene oxide chain length compared to the polyethylene oxide chain length of the second emulsifier. In a non-limiting example, the first emulsifier may be steareth-2 and the second emulsifier may be steareth-21.

The first emulsifier may be present in an amount that is greater than the second emulsifier. In some embodiments, the first and second emulsifiers may be present in a weight ratio ranging from about 1.1:1.0 to about 3.0:1.0—including all ratios and sub-ranges there-between. In some embodiments, the first and second emulsifiers may be present in a weight ratio ranging from about 1.5:1.0 to about 2.5:1.0—including all ratios and sub-ranges there-between. In a preferred embodiment, the first and second emulsifiers may be present in a weight ratio ranging from about 1.8:1.0 to about 2.2:1.0—including all ratios and sub-ranges there-between. The first and second emulsifiers may be present in a weight ratio ranging from about 2.0:1.0.

The overall amount of emulsifier may be present in an amount ranging from about 4.7 wt. % to about 10.0 wt. % based on the total weight of the deodorant composition—including all amounts and sub-ranges there-between. In a preferred embodiment, the overall amount of emulsifier may be present in an amount ranging from about 5.0 wt. % to about 9.0 wt. % based on the total weight of the deodorant composition—including all amounts and sub-ranges there-between. In a preferred embodiment, the overall amount of emulsifier may be present in an amount ranging from about 5.5 wt. % to about 8.0 wt. % based on the total weight of the deodorant composition—including all amounts and sub-ranges there-between. In a preferred embodiment, the overall amount of emulsifier may be present in an amount ranging from about 5.5 wt. % to about 7.5 wt. % based on the total weight of the deodorant composition—including all amounts and sub-ranges there-between. The overall amount of emulsifier may be present in an amount of about 6.5 wt. % based on the total weight of the deodorant composition.

The deodorant composition of the present invention may comprise a preservative. The preservative may comprise one or more compounds of sodium benzoate, lactic acid, and/or sodium lactate. in a preferred embodiment, the preservative comprises a blend of sodium benzoate, lactic acid, and sodium lactate.

The overall preservative may be present in an amount ranging from about 0.5% to about 4.5% based on the total weight of the deodorant composition—including all amounts and sub-ranges there-between. In a preferred embodiment, the preservative may be present in an amount ranging from about 1.0% to about 4% based on the total weight of the deodorant composition—including all amounts and sub-ranges there-between.

The sodium benzoate may be present in an amount ranging from about 0.15% to about 1.0% based on the total weight of the deodorant composition—including all amounts and sub-ranges there-between. The lactic acid may be present in an amount ranging from about 0.25% to about 1.5% based on the total weight of the deodorant composition—including all amounts and sub-ranges there-between. The sodium lactate may be present in an amount ranging from about 0.25% to about 2% based on the total weight of the deodorant composition—including all amounts and sub-ranges there-between. The sodium benzoate, lactic acid, and sodium lactate may be present in a weight ratio of about 1:2.25:2.25

According to the present invention, the preservative may be substantially free of phenoxyethanol. According to the present invention, the deodorant composition may be substantially free of phenoxyethanol.

The deodorant composition of the present invention may comprise one or more drying agents. The drying agent may be an inorganic compound. The drying agent may be one or more silicates—such as clay, talc, or calcium silicate. In some embodiments, the drying agent is calcium silicate. In some embodiments, the drying agent is substantially free of talc. The deodorant composition may be substantially free of talc.

The drying agent may be present in an amount ranging from about 0.1% to about 1.0% based on the total weight of the deodorant composition—including all amounts and sub-ranges there-between. In a preferred embodiment, the drying agent may be present in an amount ranging from about 0.15% to about 0.75% based on the total weight of the deodorant composition—including all amounts and sub-ranges there-between.

The deodorant composition of the present invention may further comprise one or more optional formulation additives. Other additives typically can include but are not limited to surfactants (such as foam boosters); antifoaming agents; film-formers; skin protectants; binders; other antifungal agents; opacifying agents; pearlizing agents; colorants; pigments; antioxidants, spreading aids, astringents; vitamins, fragrance solubilizers, and combinations thereof. In some embodiments, the deodorant composition may be substantially free of antioxidants. In a preferred embodiment, the deodorant composition may be substantially free of butylated hydroxytoluene.

It has been surprisingly discovered that when eliminating certain silicon-containing emollients in combination with the emulsifiers of the present invention, the resulting deodorant composition exhibits a superior separation stability while still provided the requisite odor-eliminating function required of a deodorant composition.

In some embodiments, a composition described herein is subjected to a stressing method described herein. In some embodiments, the stressing method helps to rapidly determine whether a composition is stable. In some embodiments, the methods are performed in less than 10 hours. In some embodiments, a specific G-force (or relative centrifugal force) ramp up is used to discriminate between unstable and stable compositions. In some embodiments, the methods utilize a Lumisizer analytical centrifuge manufactured by LUM Gmbh, which is controlled by SEPView software from PC. In some embodiments, the experimental conditions outlined in the Examples section are employed. In some embodiments, the separation rates (creaming rates) in % per minute at each given G-force are determined. In some embodiments, if the rate is less than ˜0.02%/min up until and including 3500 RPM (1600 G), the composition is stable. In some embodiments, the lower the number the better. In some embodiments, the methods described herein reduces the stability evaluation process from 13 weeks to 10 hours.

### EXAMPLES

A first experiment was prepared to measure the emulsion stability of the newly formulated deodorant composition. Each composition was prepared using the following components:
Crystallized alum (“Alum”)
Aluminum chlorohydrate (“ACH”)
Preservative: blend of sodium benzoate, lactic acid, sodium lactate
Emulsifier 1: a 2.1:1 ratio of Steareth-2 and Steareth-21 present in an amount of about 4.7 wt. % based on the total deodorant composition
Emulsifier 2: a 2.0:1 ratio of Steareth-2 and Steareth-21 present in an amount of about 6.57 wt. % based on the total deodorant composition

TABLE 1

| | 1 | 2 | 3 | 4 | 5 | 6 | 7 | 8 | 9 |
|---|---|---|---|---|---|---|---|---|---|
| Alum | 0% | 0% | 2% | 0% | 2% | 2% | 0% | 2% | 2% |
| ACH | 0% | 8% | 0% | 12% | 0% | 0% | 8% | 0% | 0% |
| Silicon-Containing Emollient | 0% | 0% | 0% | 0% | 0% | 0% | 2.5% | 2.5% | 0% |
| Preservative | Yes | Yes | Yes | Yes | No | Yes | No | No | Yes |
| Emulsifier 1 | No | Yes | Yes | Yes | Yes | No | Yes | Yes | Yes |
| Emulsifier 2 | Yes | No | No | No | No | Yes | No | No | No |

A more detailed ingredient listing for the formulas described in Table 1 (above), is provided below in Tables 2A and 2B (below).

TABLE 2A

| | 1 | 2 | 3 | 4 | 5 |
|---|---|---|---|---|---|
| Ingredients | Wt % | Wt % | Wt % | Wt % | Wt % |
| Water | 79.391607 | 77.424791 | 80.435318 | 74.134841 | 82.518856 |
| ACH | | 6.584420 | | 9.960840 | |
| Glycerin | 4.210105 | 4.209579 | 4.210105 | 4.209579 | 3.999600 |
| PPG-15 Stearyl Ether | 6.271578 | 6.265830 | 6.271578 | 6.275760 | 5.958000 |
| Emulsifier | 4.512316 | 3.336300 | 3.336300 | 3.336300 | 3.168000 |
| Emulsifier | 2.261369 | 1.564200 | 1.564200 | 1.564200 | 1.485000 |
| Fragrance | 0.315789 | | | | 1.100000 |
| Caprylyl Glycol | 0.510920 | 0.404880 | 0.510920 | 0.308480 | 0.482000 |
| Lactic Acid | 0.947369 | | 0.947369 | | |
| Sodium Lactate | 0.947368 | | 0.947368 | | |
| Sodium Benzoate | 0.421052 | | 0.421052 | | |
| Calcium Silicate | 0.210526 | 0.210000 | 0.210526 | 0.210000 | 0.200000 |
| Alum | | | 1.145263 | | 1.088544 |
| Total | 100 | 100 | 100 | 100 | 100 |

TABLE 2B

| | 6 | 7 | 8 | 9 |
|---|---|---|---|---|
| Ingredients | Wt % | Wt % | Wt % | Wt % |
| Water | 78.562133 | 77.118606 | 81.846642 | 80.213552 |
| ACH | | 6.415655 | | |
| Glycerin | 4.210105 | 3.999100 | 3.999100 | 3.999600 |
| PPG-15 Stearyl Ether | 6.271578 | 3.471528 | 3.471528 | 5.958000 |
| Emulsifier | 4.512316 | 3.169485 | 3.169485 | 3.169485 |
| Silicone | | 1.953385 | 1.953385 | |
| Emulsifier | 2.261369 | 1.485990 | 1.485990 | 1.485990 |
| Fragrance | | 1.200000 | 1.100000 | 1.100000 |
| Silicone | | 0.503500 | 0.503500 | |
| Caprylyl Glycol | 0.510920 | 0.384636 | 0.485374 | 0.485374 |
| Talc | | 0.169575 | 0.169575 | |
| Antioxidant | | 0.047500 | 0.047500 | |
| Silicone | | 0.040090 | 0.040090 | |
| Minerals | | 0.015960 | 0.015960 | |
| Silicone | | 0.009020 | 0.009020 | |
| Calcium Mg Carbonate | | 0.007980 | 0.007980 | |
| Magnesium Carbonate | | 0.003990 | 0.003990 | |
| Silicone | | 0.002005 | 0.002005 | |
| Silica | | 0.001995 | 0.001995 | |
| Lactic Acid | 0.947369 | | | 0.900000 |
| Sodium Lactate | 0.947368 | | | 0.900000 |
| Sodium Benzoate | 0.421052 | | | 0.400000 |
| Calcium Silicate | 0.210526 | | | 0.200000 |
| Alum | 1.145263 | | 1.088381 | 1.088000 |
| Zinc Oxide | | | | 0.100000 |
| Phenoxyethanol | | | 0.598500 | |
| Total | 100 | 100 | 100 | 100 |

Each liquid sample was then analyzed in Lumisizer, applying G-force ramp up method as set forth in Table 3 (below).

TABLE 3

| | | | | | Temperature |
|---|---|---|---|---|---|
| | | | | G-force | [° C.] |
| 1 | 12 | 5 | 1000 | 133 | 40 |
| 1 | 12 | 5 | 1413 | 266 | 40 |
| 1 | 12 | 5 | 1732 | 399 | 40 |
| 1 | 20 | 3 | 2000 | 532 | 40 |
| 0.5 | 15 | 2 | 2500 | 831 | 40 |
| 0.5 | 15 | 2 | 3000 | 1197 | 40 |
| 0.5 | 15 | 2 | 3500 | 1630 | 40 |
| 4 | 120 | 2 | 4000 | 2128 | 40 |
| Total = 9.5 | Total = 221 | | | | |

The creaming profile was recorded for each composition. The creaming rates at all segments were calculated from the profiles to determine the rate of separation within each composition—the results are set forth in Table 4 (below).

TABLE 4

| G-force | 133 | 266 | 399 | 532 | 831 | 1197 | 1630 | 2128 |
|---|---|---|---|---|---|---|---|---|
| 1 | −0.00090 | −0.00040 | 0.00030 | 0.00000 | −0.00050 | −0.00060 | 0.00020 | 0.01580 |
| 2 | −0.00140 | −0.00010 | −0.00050 | −0.00050 | 0.00050 | 0.00000 | 0.00140 | 0.02170 |
| 3 | −0.00050 | 0.00010 | −0.00080 | −0.00080 | −0.00100 | −0.00010 | 0.01640 | 0.03900 |
| 4 | −0.00020 | −0.00020 | −0.00100 | −0.00040 | 0.00090 | −0.00180 | −0.00070 | 0.02290 |
| 5 | 0.00110 | −0.00080 | −0.00080 | 0.00020 | 0.00020 | 0.00080 | 0.01670 | 0.03900 |
| 6 | −0.00040 | −0.00090 | −0.00030 | −0.00040 | −0.00020 | −0.00160 | −0.00110 | 0.02580 |
| 7 | −0.00070 | −0.00100 | −0.00090 | 0.00010 | −0.00050 | 0.00110 | 0.01760 | 0.03970 |
| 8 | −0.00270 | −0.00060 | −0.00060 | −0.00040 | 0.01660 | 0.03060 | 0.04130 | 0.04880 |
| 9 | −0.00070 | −0.00030 | 0.00070 | 0.00490 | 0.03330 | 0.04990 | 0.06440 | 0.10290 |

As demonstrated in the last column of Table 4 (above), the deodorant composition 1, of the present invention, exhibits the lowest rate of phase separation of all test samples.

Additionally, duplicates of each sample were also placed into respective containers and allowed to separate under natural gravitational conditions at 40° C. for 13 weeks, whereby the separation for each composition under normal gravity was compared against the predicted separation under centrifuge. The 3500 RPM rapid separation rates correlated very well with the 13 weeks slow method. Therefore, it can be used to predict shelf life stability.

TABLE 5

| | Absolute | Instability |
|---|---|---|
| Time | Separation | Index |
| 1 | 1.99 | 0.000 |
| 2 | 3.68 | 0.000 |
| 3 | 9.69 | 0.013 |
| 4 | 5.05 | 0.000 |
| 5 | 8.30 | 0.013 |
| 6 | 5.07 | 0.000 |
| 7 | 8.71 | 0.014 |
| 8 | 12.14 | 0.040 |
| 9 | 21.82 | 0.076 |

Further to the rapid stability prediction method by Lumisizer, it has been discovered that the %/min creaming rate calculation per each segment can be substituted by more simple Instability Index functionality within the SEPView application. More specifically, the Instability Index, as described in Table 5 (above), is determined at 350 minutes of the experiment. Thus, the threshold for shelf life stability prediction is considered an index value of ˜0.015, the lower the better.

As described in Table 5 (above), not only was the rate of separation the lowest for deodorant composition 1, but the absolution separation for composition 1 was also the lowest for all test samples.

A second experiment was prepared to measure the aesthetic properties of the deodorant composition of the present invention against a silicon-containing deodorant composition. A deodorant composition comprising 6 wt. % of PPG-15 stearyl ether (composition 10) and a Control composition comprising silicon contain emollients were each applied to underarm skin. The results are as follows:

Table 6 (below) sets forth coolness underarm measurements over time.

TABLE 6

| | Control | 10 |
|---|---|---|
| 0 Min | 5.8 | 5.7 |
| 2 Min | 2.2 | 1.9 |
| 5 Min | 0.1 | 0.1 |

As demonstrated by Table 6 (above), the new deodorant composition that is free of silicon-containing emollients performed just as well as the silicon-containing control deodorant composition for coolness.

Table 7 sets forth whiteness underarm measurements over time.

TABLE 7

| | Control | 10 |
|---|---|---|
| 0 Min | 2.3 | 2.9 |
| 2 Min | 0.6 | 1.3 |
| 5 Min | 0.1 | 0.1 |
| 10 Min | 0.0 | 0.1 |
| 15 Min | 0.0 | 0.0 |

As demonstrated the data described in Table 7 (above), after about 5 minutes, the new deodorant composition performed just as well as the silicon-containing control deodorant composition for whiteness.

Table 8 (below) sets forth stickiness underarm measurements over time.

TABLE 8

| | Control | 10 |
|---|---|---|
| 0 Min | 0.3 | 0.3 |
| 2 Min | 0.6 | 0.7 |
| 5 Min | 1.0 | 0.9 |
| 10 Min | 0.9 | 0.9 |
| 15 Min | 0.7 | 0.7 |
| 20 Min | 0.6 | 0.5 |
| 25 Min | 0.5 | 0.4 |
| 30 Min | 0.4 | 0.4 |

As demonstrated by the data described in Table 8 (above), the new deodorant composition performed better than the silicon-containing control deodorant composition for stickiness.

Table 9 sets forth wetness underarm measurements over time.

TABLE 9

| | Control | 10 |
|---|---|---|
| 0 Min | 8.9 | 8.9 |
| 2 Min | 4.8 | 4.7 |
| 5 Min | 0.6 | 0.6 |
| 10 Min | 0.0 | 0.1 |
| 15 Min | 0.1 | 0.0 |
| 20 Min | 0.0 | 0.0 |
| 25 Min | 0.0 | 0.0 |
| 30 Min | 0.0 | 0.0 |

As demonstrated by the data described in Table 9 (above), the new deodorant composition performed just as good if not better than the silicon-containing control deodorant composition for wetness.

Table 10 (below) sets forth slip underarm measurements over time.

TABLE 10

| | Control | 10 |
|---|---|---|
| 0 Min | 11.1 | 11.0 |
| 2 Min | 9.9 | 9.9 |
| 5 Min | 8.7 | 8.9 |
| 10 Min | 7.9 | 8.2 |
| 15 Min | 7.7 | 7.6 |
| 20 Min | 7.4 | 7.3 |
| 25 Min | 7.2 | 7.1 |
| 30 Min | 7.1 | 7.0 |

As demonstrated by the data described in Table 10 (above), the new deodorant composition performed as good as the silicon-containing control deodorant composition for slip.

Table 11 (below) sets forth oily residue underarm measurements over time.

TABLE 11

| | Control | 10 |
|---|---|---|
| 0 Min | 8.9 | 8.9 |
| 2 Min | 5.3 | 5.5 |
| 5 Min | 2.1 | 2.6 |
| 10 Min | 0.9 | 1.0 |
| 15 Min | 0.5 | 0.4 |
| 20 Min | 0.1 | 0.1 |
| 25 Min | 0.0 | 0.0 |
| 30 Min | 0.0 | 0.0 |

As demonstrated by the data described in Table 11 (above), the new deodorant composition performed about equal to the silicon-containing control deodorant composition for oily residue.

Table 12 (below) describes the powder underarm measurements over time.

TABLE 12

| | Control | 10 |
|---|---|---|
| 0 Min | 0.0 | 0.0 |
| 2 Min | 0.0 | 0.0 |
| 5 Min | 0.0 | 0.0 |
| 10 Min | 0.0 | 0.0 |
| 15 Min | 0.0 | 0.0 |
| 20 Min | 0.0 | 0.0 |
| 25 Min | 0.0 | 0.0 |
| 30 Min | 0.0 | 0.0 |

As demonstrated by the data described in Table 12 (above), the new deodorant composition performed equal to the silicon-containing control deodorant composition for powder.

Table 13 (below) sets forth pilling underarm measurements over time.

TABLE 13

| | Control | 10 |
|---|---|---|
| 0 Min | 0.0 | 0.0 |
| 2 Min | 0.0 | 0.0 |
| 5 Min | 0.0 | 0.0 |
| 10 Min | 0.0 | 0.0 |
| 15 Min | 0.0 | 0.0 |
| 20 Min | 0.0 | 0.0 |
| 25 Min | 0.0 | 0.0 |
| 30 Min | 0.0 | 0.0 |

As demonstrated by the data described in Table 13 (above), the new deodorant composition performed equal to the silicon-containing control deodorant composition for pilling.

Table 14 (below) sets forth fragrance underarm measurements over time.

TABLE 14

| | Control | 10 |
|---|---|---|
| 0 Min | 6.8 | 6.9 |
| 2 Min | 6.3 | 6.2 |
| 5 Min | 5.7 | 5.6 |
| 10 Min | 5.4 | 5.1 |
| 15 Min | 4.7 | 4.7 |
| 20 Min | 4.4 | 4.4 |
| 25 Min | 3.9 | 4.1 |
| 30 Min | 3.9 | 3.7 |

As demonstrated by data described in Table 14 (above), the new deodorant composition performed about the same as the silicon-containing control deodorant composition for powder.

1. A personal care composition comprising:
an odor-reducing agent comprising a non-aluminum compound;
an emollient comprising a non-volatile ether compound;
calcium silicate; and
an emulsifier.

2. The personal care composition according to claim 1, wherein the non-volatile ether compound comprises an ether of stearyl alcohol.

3. The personal care composition according to claim 2, wherein the ether of stearyl alcohol is polypropylene glycol stearyl ether.

4. The personal care composition according to claim 3, wherein the polypropylene glycol stearyl ether is a PPG-15 stearyl ether.

5. The personal care composition according to claim 1, wherein the emollient is substantially free of volatile compounds.

6. The personal care composition according to claim 1, where the emollient is substantially free of cyclomethicone and dimethicone.

7.-8. (canceled)

9. The personal care composition according to claim 1, further comprising a preservative that is substantially free of phenoxyethanol.

10. (canceled)

11. The personal care composition according to claim 1, wherein the deodorant composition is substantially free of talc.

12.-13. (canceled)

14. The personal care composition according to claim 1, wherein the odor-reducing agent is substantially free of potassium alum and aluminum chlorohydrate.

15.-19. (canceled)

20. The personal care composition according to claim 1, wherein the personal care composition is substantially free of butylated hydroxytoluene.

21. The personal care composition according to claim 20, wherein the personal care composition is substantially free of antioxidant.

22.-23. (canceled)

24. A deodorant composition comprising:
an odor-reducing agent;
an emollient that is substantially free of cyclomethicone and dimethicone;
an emulsifier comprising a blend of two or more different stearyl ether; and
wherein the emulsifier is present in an amount ranging from about 5.5 wt. % to about 8.0 wt. % based on the total weight of the deodorant composition.

25. The deodorant composition according to claim 24, wherein the emulsifier comprises a blend of steareth-2 and steareth-21.

26. The deodorant composition according to claim 25, wherein the steareth-2 and steareth-21 are present in a weight ratio ranging from about 1.5:1 to about 2.5:1.

27.-32. (canceled)

33. The deodorant composition according to claim 24, wherein the odor-reducing agent is substantially free of potassium alum.

34. The deodorant composition according to claim 24, wherein the odor-reducing agent is substantially free of aluminum chlorohydrate.

35.-37. (canceled)

38. The deodorant composition according to claim 24, further comprising a preservative that is substantially free of phenoxyethanol.

39. (canceled)

40. The deodorant composition according to claim 24, wherein the deodorant composition is substantially free of butylated hydroxytoluene.

41. The deodorant composition according to claim 40, wherein the deodorant composition is substantially free of antioxidant.

42. A method of forming a separation stable personal care composition, the method comprising:
a) forming a blend of
an odor-reducing agent comprising a non-aluminum compound;
an emollient that is substantially free of cyclomethicone and dimethicone;
an emulsifier comprising a blend of a first stearyl ether and a second stearyl ether, wherein the first and second stearyl ethers are different and the first stearyl ether is present in an amount greater than the second stearyl ether, and the emulsifier is present in an amount ranging from about 5.5 wt. % to about 8.0 wt. % based on the total weight of the blend;
wherein the blend has a viscosity ranging from about a viscosity in the range of 1500 mPas to about 4000 mPas.

43.-88. (canceled)
